(19) United States
(12) Patent Application Publication
(10) Pub. No.: US 2016/0288974 A1
(43) Pub. Date: Oct. 6, 2016

(54) CARTRIDGE HUB PACKAGING

(71) Applicant: SANOFI-AVENTIS DEUTSCHLAND GMBH, Frankfurt am Main (DE)

(72) Inventors: Marc Holtwick, Frankfurt am Main (DE); Jens Schnur, Beerfelden / Olfen (DE); Andreas Krämmerer, Stadecken-Elsheim (DE); Gilbert Robel, Oberursel (DE)

(21) Appl. No.: 15/035,186

(22) PCT Filed: Nov. 19, 2014
(86) PCT No.: PCT/EP2014/075057

§ 371 Date: May 6, 2016

(30) Foreign Application Priority Data
Nov. 21, 2013 (EP) 13193840.9

(51) Int. Cl. B65D 75/32; A61M 5/00; B65D 75/36; B65D 25/10

(52) U.S. Cl. CPC B65D 75/326; B65D 25/108; A61M 5/002; B65D 75/366

(57) ABSTRACT

The invention relates to a blister package (500, 700, 800, 1000, 1100, 1200, 1320, 1420) for packaging a dispense interface (200, 2000) of a medicament ejection device, with a blister (510, 710, 810, 1010, 1110, 1210) having a cavity portion (512, 712, 812, 1012, 1112, 1212) and a flange (514, 1014, 1214), the cavity portion (512, 712, 812, 1012, 1112, 1212) defining a cavity (516) and being configured to hold a dispense interface (200, 2000) of a medicament ejection device within the cavity (516), and with a lid (520, 720, 1020, 1120, 820) bonded to the flange (514, 1014, 1214) of said blister (510, 710, 810, 1010, 1110, 1210), wherein the cavity portion (512, 712, 812, 1012, 1112, 1212) of said blister (510, 710, 810, 1010, 1110, 210) comprises at least one undercut portion configured to interact with a corresponding part of a dispense interface (200, 2000) to be packaged in the blister package (500, 700, 800, 1000, 1100, 1200, 1320, 1420) to lock the dispense interface (200, 2000) within the cavity (516). The invention also relates to a system comprising a dispense interface (200, 2000) of a medicament ejection device and a blister package (500, 700, 800, 1000, 1100, 200, 1320, 1420) as described before, wherein the blister package (500, 700, 800, 1000, 100, 1200, 1320, 1420) is adapted to the dispense interface (200, 2000).

The present invention inter alia relates to a blister package for packaging a dispense interface of a medicament ejection device with a blister having a cavity portion and a flange, the cavity portion defining a cavity and being configured to hold a dispense interface of a medicament ejection device within the cavity, and with a lid bonded to the flange of said blister. The invention further relates to a system comprising a dispense interface of a medicament ejection device and a blister package as described above. The dispense interface of a medicament ejection device may be a part of a medical device, in particular a disposable part of a medical device, which is to be assembled with another part of the medical device for operation.

The medical device can be an injector, for example a hand-held injector, especially a pen-type injector, that is an injector of the kind that provides for administration by injection of medicinal products from one or more multidose cartridges. In particular, the present invention relates to such injectors where a user may set the dose.

The drug agents may be contained in two or more multiple dose reservoirs, containers or packages, each containing independent (single drug compound) or pre-mixed (co-formulated multiple drug compounds) drug agents.

Certain disease states require treatment using one or more different medicaments. Some drug compounds need to be delivered in a specific relationship with each other in order to deliver the optimum therapeutic dose. The present patent application is of particular benefit where combination therapy is desirable, but not possible in a single formulation for reasons such as, but not limited to, stability, compromised therapeutic performance and toxicology.

For example, in some cases it may be beneficial to treat a diabetic with a long acting insulin (also may be referred to as the first or primary medicament) along with a glucagon-like peptide-1 such as GLP-1 or GLP-1 analog (also may be referred to as the second drug or secondary medicament).

Accordingly, there exists a need to provide devices for the delivery of two or more medicaments in a single injection or delivery step that is simple for the user to perform without complicated physical manipulations of the drug delivery device. The proposed drug delivery device provides separate storage containers or cartridge retainers for two or more active drug agents. These active drug agents are then combined and/or delivered to the patient during a single delivery procedure. These active agents may be administered together in a combined dose or alternatively, these active agents may be combined in a sequential manner, one after the other.

The drug delivery device also allows for the opportunity of varying the quantity of the medicaments. For example, one fluid quantity can be varied by changing the properties of the injection device (e.g., setting a user variable dose or changing the device's “fixed” dose). The second medicament quantity can be changed by manufacturing a variety of secondary drug containing packages with each variant containing a different volume and/or concentration of the second active agent.

The drug delivery device may have a single dispense interface. This interface may be configured for fluid communication with a primary reservoir and with a secondary reservoir of medicament containing at least one drug agent. The drug dispense interface can be a type of outlet that allows the two or more medicaments to exit the system and be delivered to the patient.

The combination of compounds from separate reservoirs can be delivered to the body via a double-ended needle assembly. This provides a combination drug injection system that, from a user's perspective, achieves drug delivery in a manner that closely matches the currently available injection devices that use standard needle assemblies. One possible delivery procedure may involve the following steps:

1. Attach a dispense interface to a distal end of the electro-mechanical injection device. The dispense interface comprises a first and a second proximal needle. The first and second needles pierce a first reservoir containing a primary compound and a second reservoir containing a secondary compound, respectively.

2. Attach a dose dispenser, such as a double-ended needle assembly, to a distal end of the dispense interface. In this manner, a proximal end of the needle assembly is in fluidic communication with both the primary compound and secondary compound.

3. Dial up/set a desired dose of the primary compound from the injection device, for example, via a graphical user interface (GUI).

4. After the user sets the dose of the primary compound, the micro-processor controlled control unit may determine or compute a dose of the secondary compound and preferably may determine or compute this second dose based on a previously stored therapeutic dose profile. It is this computed combination of medicaments that will then be injected by the user. The therapeutic dose profile may be user selectable. Alternatively, the user can dial or set a desired dose of the secondary compound.

5. Optionally, after the second dose has been set, the device may be placed in an armed condition. The optional armed condition may be achieved by pressing and/or holding an “OK” or an “Arm” button on a control panel. The armed condition may be provided for a predefined period of time during which the device can be used to dispense the combined dose.

6. Then, the user will insert or apply the distal end of the dose dispenser (e.g. a double ended needle assembly) into the desired injection site. The dose of the combination of the primary compound and the secondary compound (and potentially a third medicament) is administered by activating an injection user interface (e.g. an injection button).

Both medicaments may be delivered via one injection needle or dose dispenser and in one injection step. This offers a convenient benefit to the user in terms of reduced user steps compared to administering two separate injections.

There is a general desire to improve drug delivery devices such as the drug delivery device described above in order to prevent contamination of the drugs to be administered with the device. Contamination may for example occur if a volume of a drug remains in a dead volume of a drug delivery device, such as inside a dispense interface, for a longer time. Furthermore, cross-contamination of for example two drugs to be administered with the same drug delivery device may occur when a volume of each of the two drugs remain, for example, in a dispense interface for a longer time.

In order to prevent contamination or cross-contamination of the drug or drugs, dispense interfaces like the dispense interface of the drug delivery device described above are often laid out as disposable parts. Such a dispense interface is then only laid out or designated for a limited number of drug injections, for example for only one drug ejection, before the used dispense interface has to be or shall be replaced by a fresh dispense interface. In this way the risk of contamination and cross-contamination may be reduced by exchanging a used dispense interface by a new one.

To this end, the user however usually has to keep ready and/or store at least one or more fresh dispense interfaces so that he is able to mount a fresh dispense interface to the drug delivery device. This may cause additional risks for contamination if sterility of the fresh dispense interface cannot be ensured before mounting it on the drug delivery device. Furthermore, dispense interfaces often comprise sharp elements such as needles or cannulas so that there may be a risk of injury for the user when storing, handling or disposing the dispense interfaces or there may also be a risk of damaging the dispense interfaces.

In view of the aforementioned it is an object of the present invention to provide a suitable package for securely packaging a dispense interface for a medicament ejection device. In particular it is an object of the present invention to store a dispense interface for a medicament ejection device in a manner so that it is not contaminated or damaged during storing. It is further an object of the present invention to store a dispense interface for a medicament ejection device in a manner so that the risk of injury is reduced for the user. A further object of the present invention is also to provide an according system comprising a dispense interface, of a medicament ejection device and an according package.

Said dispense interface may for example be a dispense interface comprising a main body part with a main body proximal end and a main body distal end, wherein a mounting hub is provided at the distal end of the main body part, which mounting hub may comprise an outer thread configured to interact with an inner thread of a needle hub of a needle assembly. In particular, the main body of the dispense interface may provide gripping grooves to improve handling of the dispense interface.

According to a first aspect of the present invention, the objects described above are at least in part solved by a blister package for packaging a dispense interface of a medicament ejection device with a blister having a cavity portion and a flange, the cavity portion defining a cavity and being configured to hold a dispense interface of a medicament ejection device within the cavity, and with a lid bonded to the flange of said blister, wherein the cavity portion of said blister comprises at least one undercut portion configured to interact with a corresponding part of a dispense interface to be packaged in the blister package to lock the dispense interface within the cavity.

The blister package comprises a blister having a cavity portion and a flange. The blister may for example be made of a plastic, in particular of a transparent plastic so that the dispense interface packaged within the blister is visible from the outside. For example, the blister may be manufactured by injection molding/vacuum forming. The cavity portion defines a cavity and is configured to hold a dispense interface of a medicament ejection device within the cavity. This is understood to mean, that the size and the shape of the cavity is configured such that a dispense interface of a medicament ejection device may be accommodated in the cavity. Preferably, the inner shape of the cavity is adapted to the outer shape of the dispense interface of the medicament ejection device to be packaged in the blister package, so that on the one hand said dispense interface fully fits into the cavity and on the other hand the blister does not occupy unnecessary void space and allows space-saving storing. The blister may comprise undercuts or other locking features that are formed by the inner shape of the blister to the outer shape of the dispense interface. Preferably, the undercuts or locking features are formed in such a way that the forces holding the dispense interface in the blister can be easily overcome by a user when using the device to pick up the dispense interface from the blister.

The flange of the blister package is preferably arranged around an opening of the cavity portion through which a dispense interface may be inserted into the cavity portion or taken out of it. The flange may for example have essentially the shape of a closed loop.

A lid is bonded to the flange of said blister, for example by means of an adhesive such as glue. Preferably, the lid seals the opening of the cavity portion around which the flange is arranged. The lid may be made of plastic, of a metal material or a combination thereof. Preferably, the lid material and thickness is selected such to prevent bacteria and other microorganisms to penetrate through the lid into the cavity so that a dispense interface accommodated within the cavity portion is kept sterile. Preferably, the volume within the cavity portion is hermetically sealed from the outside by means of the lid.

The cavity portion of the blister comprises at least one undercut portion configured to interact with a corresponding part of the dispense interface to be packaged in the blister package to lock the dispense interface within the cavity. An undercut portion is a portion of the cavity portion that comprises an undercut with reference to an opening through which the dispense interface is taken out or inserted into the cavity portion. This opening may in particular be the opening that is surrounded by the flange and preferably sealed by the lid bonded to the flange.

It was found that the dispense interface may be stored within the blister package more securely by providing at least one such undercut portion to hold the dispense interface in place within the cavity portion. In this way, the dispense interface may be prevented from falling out of the cavity if—for example—the user has inadvertently removed the lid. Moreover, the dispense interface and in particular sharp elements thereof are hold back from the lid when—for example—during transport the blister package is oriented bottom-up. This may prevent sharp elements of the dispense interface to perforate the lid, so that sterility of the dispense interface may be preserved.

According to a first embodiment the cavity portion comprises a lateral portion configured to laterally enclose a main body part of a dispense interface to be packaged in the blister package and two opposing faces of the lateral portion each comprise at least one snap-fit, more preferably at least two snap-fits, configured to interact with a proximal end of the dispense interface.

It was found that by providing two opposing snap-fits, i.e. at least one snap-fit on two opposing faces of the lateral portion of the cavity portion, the dispense interface may be securely locked within the cavity in the direction of the lid or the opening sealed by the lid, respectively. Furthermore such snap-fits may be manufactured relatively easily during production of the blister, for example during injection molding the blister.

According to a further embodiment the cavity portion comprises a lateral portion configured to laterally enclose a main body part of a dispense interface to be packaged in the blister package and two opposing faces of the lateral portion each comprise at least one undercut portion configured to interact with corresponding gripping grooves on the main body part of the dispense interface.

Dispense interfaces of medicament ejection devices often comprise gripping grooves to ease handling of the dispense interface for the user. Such gripping grooves are typically provided on two opposing outer sides of the dispense interface. By providing at least one undercut portion configured to interact with these corresponding gripping grooves of the dispense interface, the gripping grooves may be used to lock the dispense interface within the cavity.

According to a further embodiment the undercut portion of the cavity portion comprises a bulge having a first face facing the lid and a second face facing away from the lid, said first face forming the undercut surface and said second face being inclined to guide a part of the dispense interface to be packaged in the blister package over the bulge. To this end, the second face is preferably inclined to the direction perpendicular to the plane of the lid, in particular with an angle of more than 20°, preferably more than 30°, in particular more than 45°. Preferably, the cavity may comprise at least two such bulges on two opposing lateral sides.

When the dispense interface is inserted into the cavity, for example during original packaging or if a user inserts a used dispense interface into the cavity for disposal, a part of the dispense interface interacts with the inclined second surface so that the bulge is elastically pushed to the side and gives way to the dispense interface to pass the bulge and be inserted completely into the cavity more easily. After the respective part of the dispense interface has passed over the bulge, the bulge then snaps back and thus locks the dispense interface within the cavity portion. The bulge, in particular the first face of the bulge, has the effect of a ramp to guide the dispense interface into a locked position within the cavity portion.

According to a second aspect of the present invention the objects described above are at least in part solved by a blister package for packaging a dispense interface of a medicament ejection device with a blister having a cavity portion and a flange, the cavity portion defining a cavity and being configured to hold a dispense interface of a medicament ejection device within the cavity, and with a lid bonded to the flange of said blister, wherein the cavity portion comprises a distal portion configured to accommodate a mounting hub of the dispense interface to be packed in the blister package and wherein the distal portion comprises radial inwardly directed clamping portions configured to interact with the mounting hub of the dispense interface, in particular with an outer thread of the mounting hub.

Dispense interfaces often comprise mounting hubs to attach thereto a needle hub of a needle assembly, which for example may be replaced after each injection. To this end, such mounting hubs may comprise an outer thread and the according needle hub may comprise a corresponding inner thread to securely attach the needle hub to the mounting hub. By means of the clamping portions of the distal portion, the mounting hub or in particular the outer thread of the mounting hub may be employed to lock the dispense interface within the cavity of a blister package so that the dispense interface is securely fixed within the package.

According to a third aspect of the present invention the objects described above are at least in part solved by a blister package for packaging a dispense interface of a medicament ejection device with a blister having a cavity portion and a flange, the cavity portion defining a cavity and being configured to hold a dispense interface of a medicament ejection device within the cavity, and with a lid bonded to the flange of said blister, wherein the blister comprises a cap portion being pivotably connected to the flange by an integral hinge so that the cap portion is movable between a closed position covering the cavity portion and an open position, and wherein the cap portion preferably comprises a reception portion configured to accommodate a mounting hub of the dispense interface to be packaged in the blister package.

The blister is preferably one-part. For example the blister with its cavity portion, flange and cap portion may be produced in one process, for example by injection molding. The cap portion is pivotably connected to the flange by an integral hinge. An integral hinge is understood to designate a hinge that is formed by a thin film being part of the blister, for example by a thin foil portion that is flexible enough to be folded so that the cap portion is movable from the closed position to the open position and vice versa.

The cap portion preferably comprises a reception portion configured to accommodate a mounting hub of the dispense interface to be packaged in the blister package. In this way, the mounting hub of said dispense interface may for example be inserted into said reception portion of the cap portion after use. This allows the user to safely store the used dispense interface within the blister package without the risk of injury due to sharp elements of the dispense interface. Preferably the reception portion comprises radial inwardly directed clamping portions to fixate the dispense interface at the cap portion.

According to a fourth aspect of the present invention the objects named above are at least in part solved by a blister package for packaging a dispense interface of a medicament ejection device with a blister having a cavity portion and a flange, the cavity portion defining a cavity and being configured to hold a dispense interface of a medicament ejection device within the cavity, and with a lid bonded to the flange of said blister, wherein the cavity portion comprises a lateral portion configured to laterally enclose a dispense interface to be packaged in the blister package, and wherein the lateral portion comprises at least two opposing faces, the outer shape of which is mirror asymmetrical.

When stacking a plurality of blister packages on top of each other to store the blister packages in a space-saving manner, an upper blister package due to its weight exerts a compression force onto a lower blister package being arranged below the upper blister package. A large portion of this compression force is often exerted onto the lid of the lower blister package which may lead to lid damaging or even to lid destruction.

It was found that the force exerted on a lid in such a situation may be reduced by providing blister packages as described above. Such blister packages with two opposing faces being mirror asymmetrical in shape may be piled up in such a way, that the upper blister package is rotated around a vertical axis by about 180° with respect to the lower blister package. For example, if the blister packages are piled up in rows, the blister packages in a first row may have a first orientation with respect to the two opposing faces and the blister packages in the row above the first row may have a second, mirrored orientation. Due to the shape asymmetry of the opposing faces the force exerted from the upper blister package onto the lower blister package may be at least in part guided into the lateral sides of the blister of the lower blister package instead of into the lid.

Preferably, the blisters of such blister packages may still be stacked within each other if all blister packages have the same orientation. This allows a space-saving storage of the blisters for example during production or after use when the lid has been removed.

According to an embodiment of the blister package the lateral portion of the cavity portion may have a substantially rectangular cross section with corresponding indentations and protrusions on two opposite faces of the lateral portion, preferably on the long side of the rectangular cross section, in order to provide for a mirror asymmetrical shape.

According to a fifth aspect of the present invention the objects described above are at least in part solved by a blister package for packaging a dispense interface of a medicament ejection device with a blister having a cavity portion and a flange, the cavity portion providing a cavity and being configured to hold a dispense interface of a medicament ejection device within the cavity, and with a lid bonded to the flange of said blister, wherein the blister comprises a bottom portion being located opposite to the lid and defining the bottom of the cavity, and wherein the blister further comprises a holding portion extending away from the lid and beyond the bottom portion.

By means of the holding portion extending away from the lid and beyond the bottom portion, the blister provides a larger surface to grab or hold the blister package, for example with a hand. In this way, handling the blister package is made easier, in particular for handicapped people, for example when holding the blister package to open the lid an taking the dispense interface out of the blister package. The holding portion extending away from the lid and beyond the bottom portion is understood to mean that the holding portion extends further than the bottom portion in a direction away from the lid. The holding portion does not necessarily need to extend from the lid, but rather in a direction away from the lid.

According to an embodiment the total height of the blister package is increased by the holding portion by at least 10%, preferably by at least 25% with respect to the height of the cavity formed by the cavity portion alone (i.e. excluding the part of the holding portion extending beyond the bottom portion).

According to a sixth aspect of the present invention the objects described above are at least in part solved by a blister package for packaging a dispense interface of a medicament ejection device with a blister having a cavity portion and a flange, the cavity portion providing a cavity and being configured to hold the dispense interface of a medicament ejection device within the cavity, and with a lid bonded to the flange of said blister, wherein the lid comprises a peel-off portion, in which the lid is less strongly bonded or unbonded to the flange and wherein the flange has a width of at least 10 mm, more preferably at least 15 mm, in particular at least 20 mm at least in the region of the peel-off portion.

By means of the peel-off portion, a user may easily grab a part of the lid in the peel-off portion and peel off the lid from the blister to open the blister package in order to take out a dispense interface from the blister package. By providing a flange with a broad width at least in the region of the peel-off portion, the peel-off portion may be increased in size so that handling of the blister package is made easier for, for example, handicapped persons. Preferably, at least 25% of the flange, more preferably at least 50% of the flange, in particular essentially the full flange has such a broad width.

According to another aspect of the invention, the objects described above are at least in part solved by a blister package for packaging a dispense interface of a medicament ejection device, wherein the blister package is according to one of the aspects or embodiments of the invention described above or any combination thereof. In particular, the features of the blister packages according to the first to the sixth aspect of the present invention may be combined with each other at will and as needed in order to achieve their respective advantages. As a non-limiting example, the first aspect of the invention may for example be combined with the fifth and sixth aspect so that by providing an undercut portion a dispense interface may be safely locked within the cavity of the blister package while at the same time handling of the blister package is made easier, in particular for handicapped persons, by a holding portion and a peel-off portion with broad flange width. One skilled in the art can of course use any other combination of the aspects of the present disclosure as described above.

The objects described above are further at least in part solved by a system comprising a dispense interface of a medicament ejection device and a blister package according to any aspect or embodiment of the invention described above or any combination thereof. Preferably, the blister package is adapted to the dispense interface. In this way a particular blister package may be adapted in its shape and configuration to a particular dispense interface, in particular to its outer dimensions. The blister package may for example be adapted to the dispense interface in that the size and geometry of the cavity provided by the cavity portion of the blister is configured to accommodate the dispense interface.

According to a further embodiment of the system the dispense interface is accommodated in the cavity provided by the cavity portion of the blister package.

According to a further embodiment of the system the dispense interface comprises a main body part with a main body proximal end and a main body distal end and a mounting hub is provided at the distal end of the main body part, which mounting hub preferably comprises an outer thread configured to interact with an inner thread of a needle hub of a needle assembly. In particular, the main body of the dispense interface may provide gripping grooves to improve handling of the dispense interface.

These as well as other advantages of various aspects of the present invention will become apparent to those of ordinary skill in the art by reading the following detailed description, with appropriate reference to the accompanying drawings, in which:

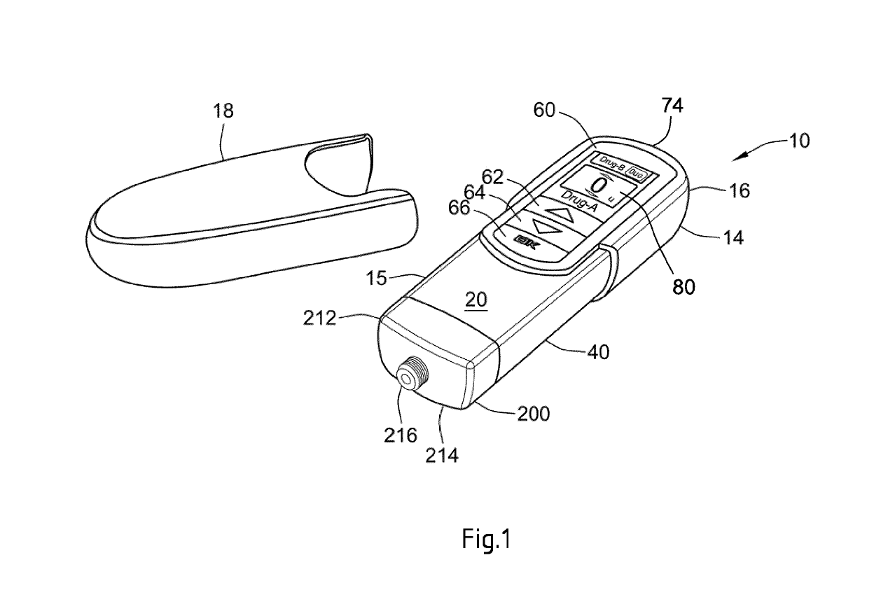

FIG. 1 illustrates a perspective view of a delivery device with an end cap of the device removed;

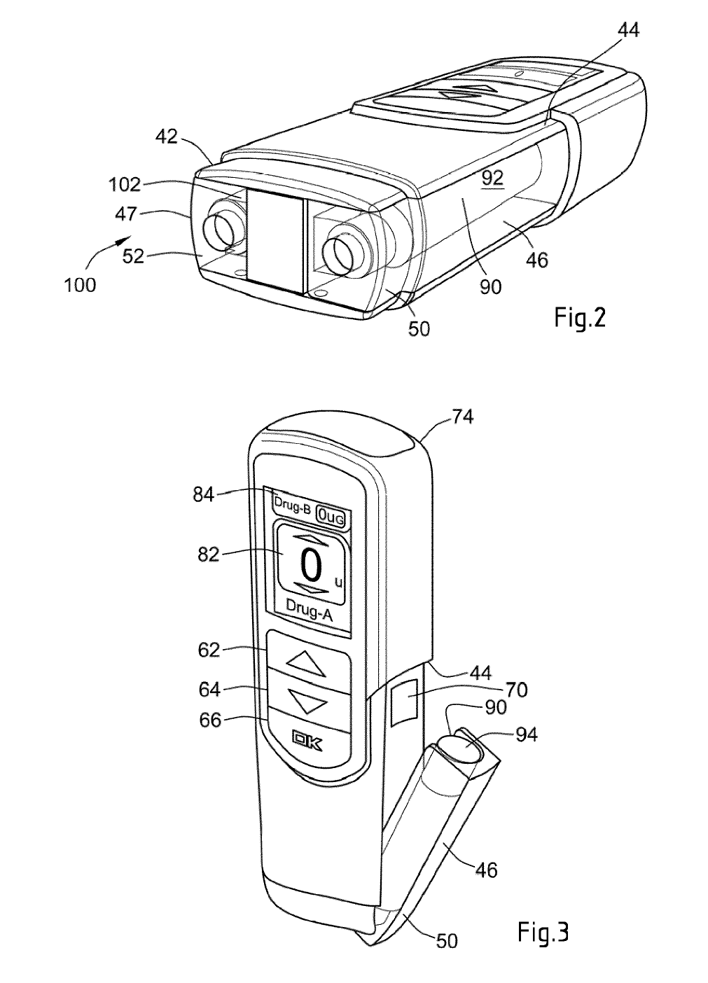

FIG. 2 illustrates a perspective view of the delivery device distal end showing the cartridge;

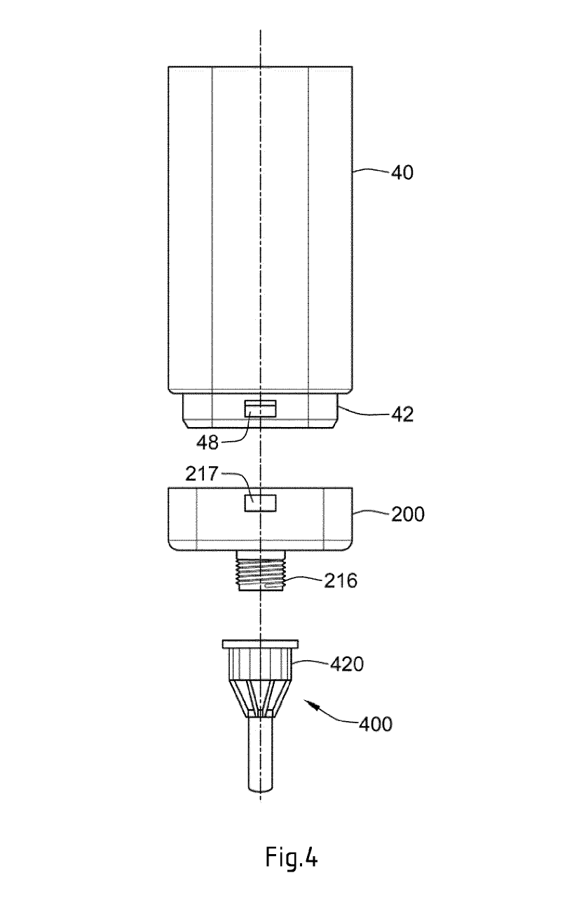

FIG. 3 illustrates a perspective view of the delivery device illustrated in FIG. 1 or 2 with one cartridge retainer in an open position;

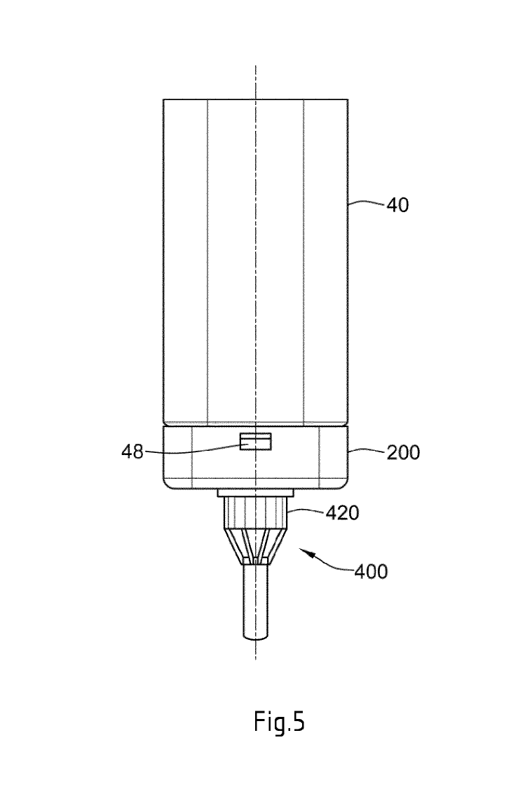

FIG. 4 illustrates a dispense interface and a dose dispenser that may be removably mounted on a distal end of the delivery device illustrated in FIG. 1;

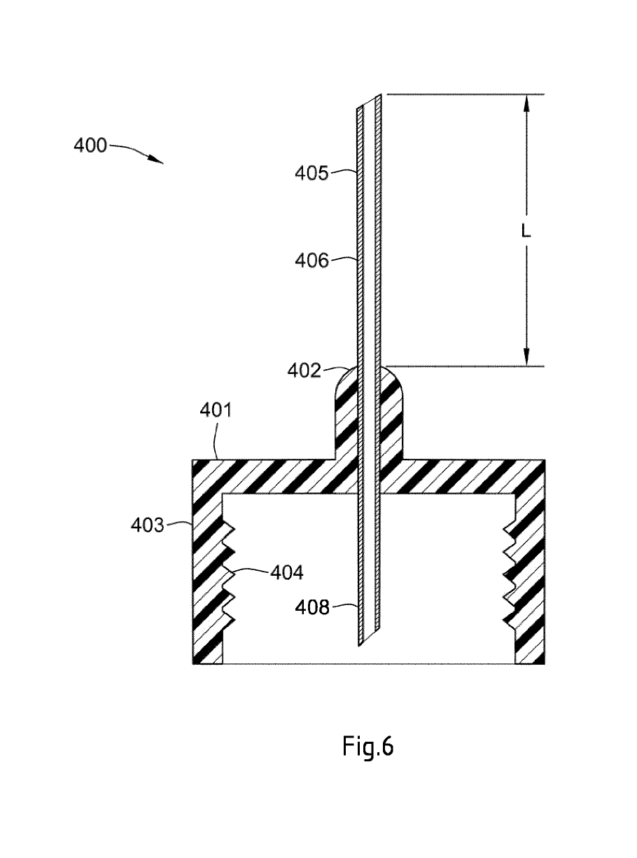

FIG. 5 illustrates the dispense interface and the dose dispenser illustrated in FIG. 4 mounted on a distal end of the delivery device illustrated in FIG. 1;

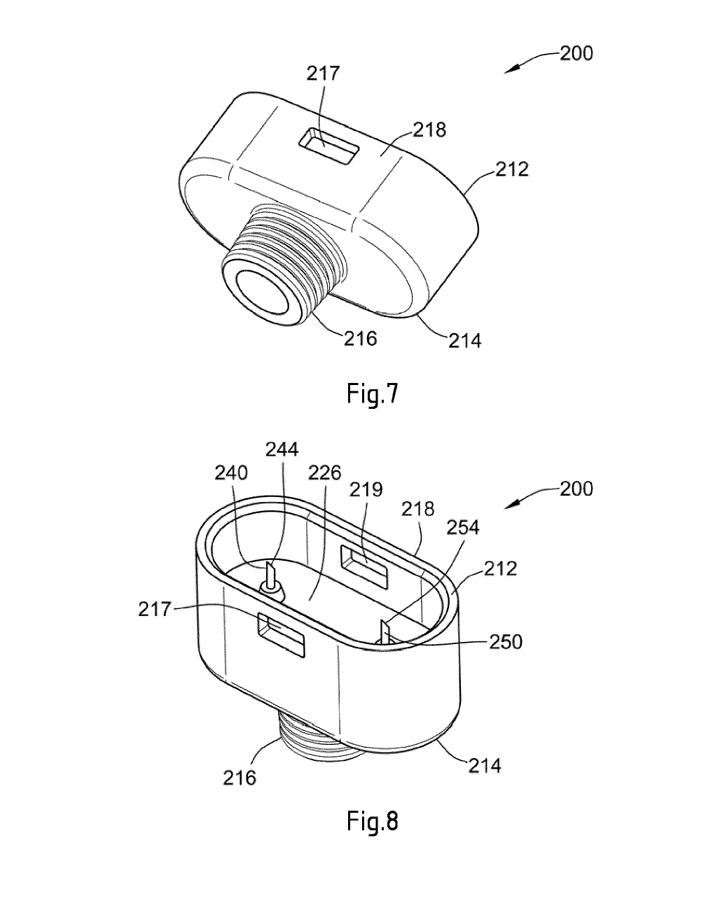

FIG. 6 illustrates one arrangement of a needle assembly that may be mounted on a distal end of the delivery device;

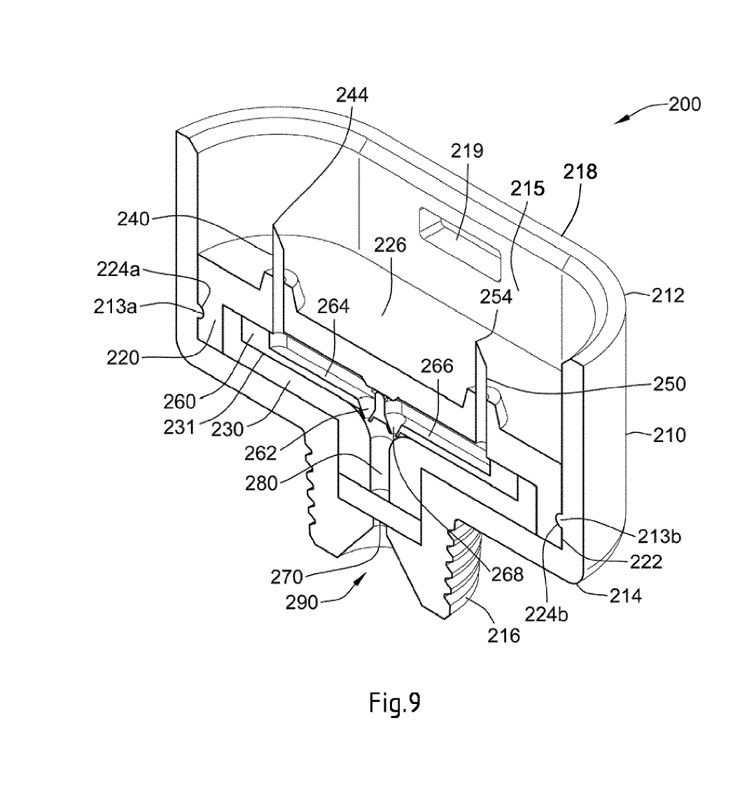

FIG. 7 illustrates a perspective view of the dispense interface illustrated in FIG. 4;

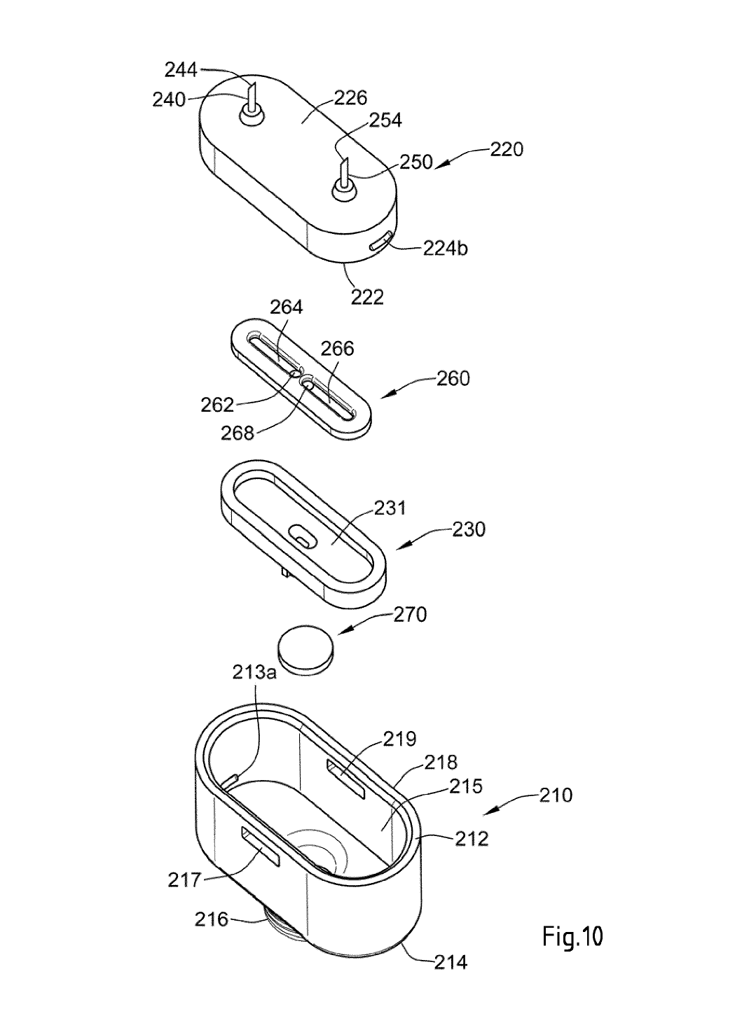

FIG. 8 illustrates another perspective view of the dispense interface illustrated in FIG. 4;

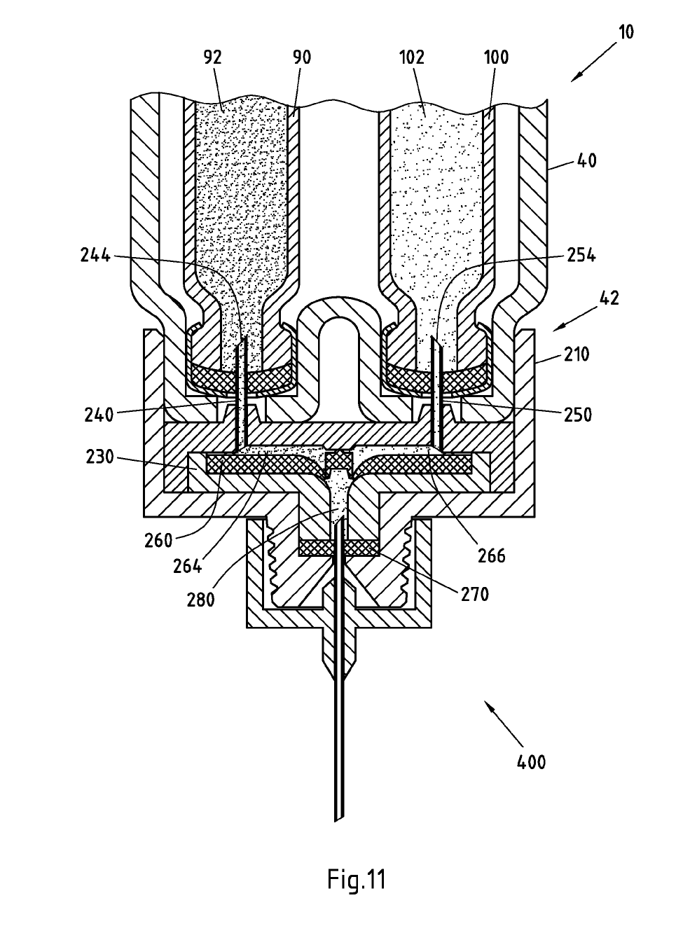

FIG. 9 illustrates a cross-sectional view of the dispense interface illustrated in FIG. 4;

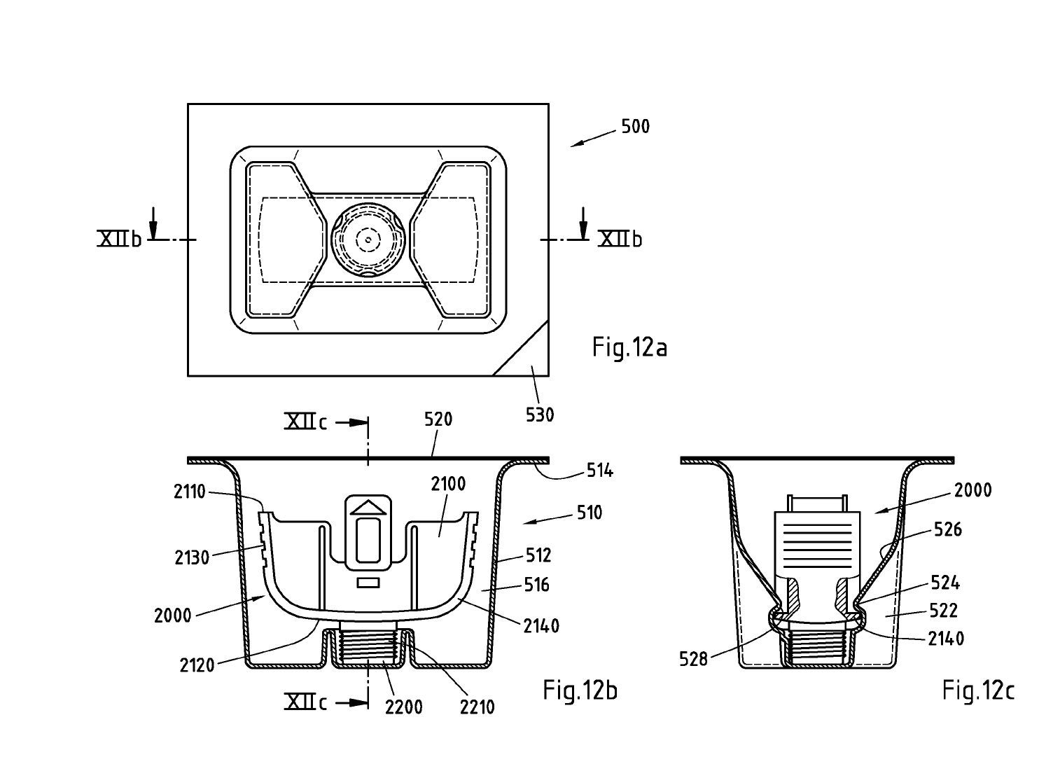

FIG. 10 illustrates an exploded view of the dispense interface illustrated in FIG. 4;

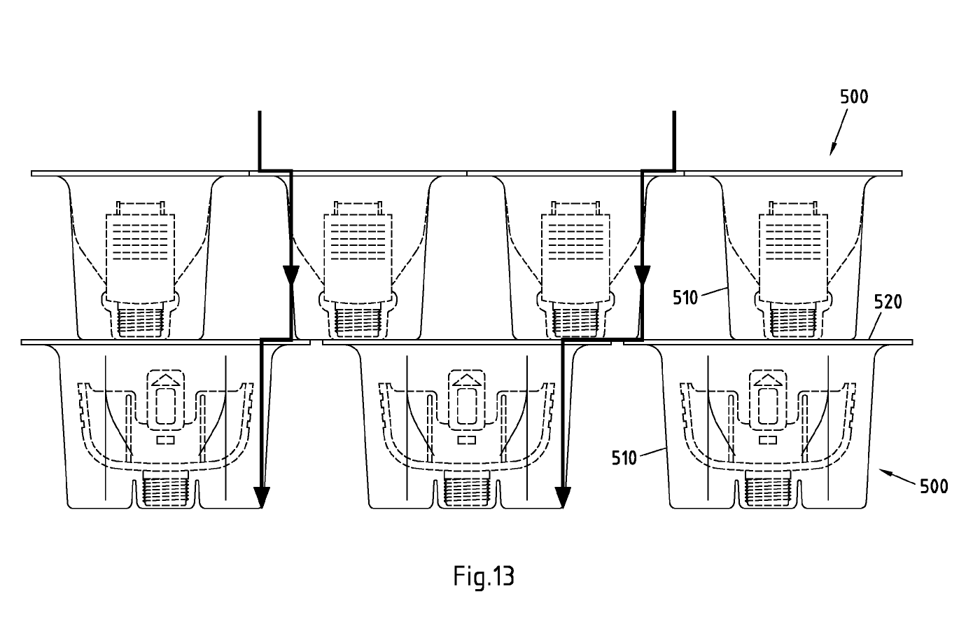

FIG. 11 illustrates a cross-sectional view of the dispense interface and needle assembly mounted onto a drug delivery device, such as the device illustrated in FIG. 1;

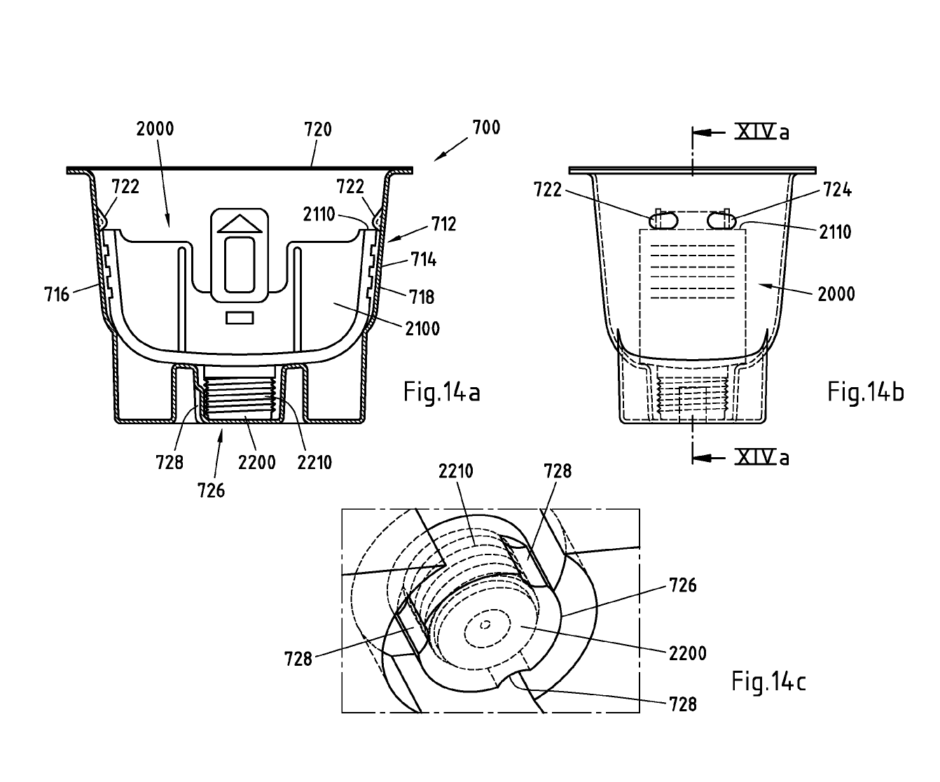

FIG. 12 a-c illustrate an exemplary embodiment of a blister package for a dispense interface according to the first aspect of the invention and a corresponding system;

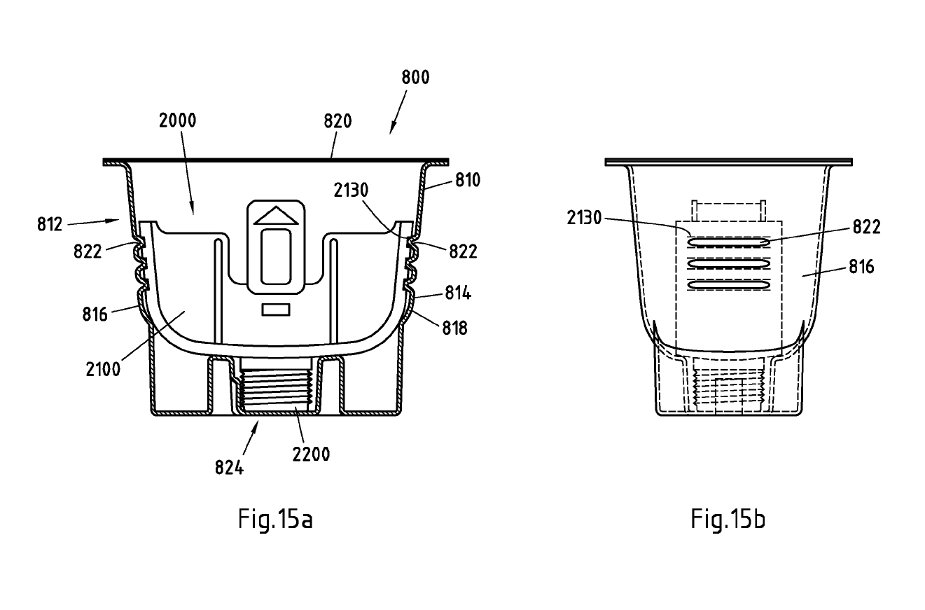

FIG. 13 illustrates a plurality of stacked blister packages from FIG. 12a-c;

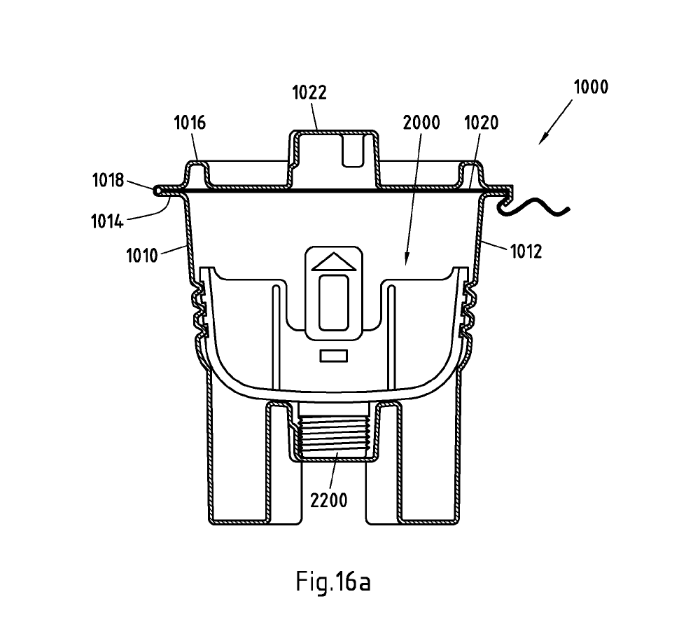

FIG. 14 a-c illustrate an exemplary embodiment of a blister package for a dispense interface according to the first and second aspect of the invention and a corresponding system;

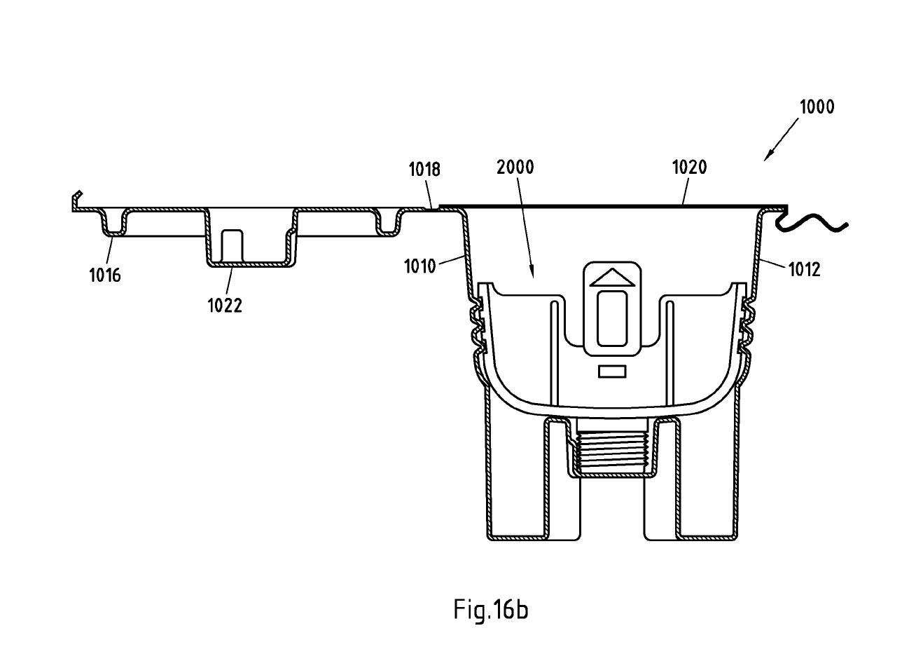

FIG. 15 a-b illustrate an exemplary embodiment of a blister package for a dispense interface according to the first and second aspect of the invention and a corresponding system;

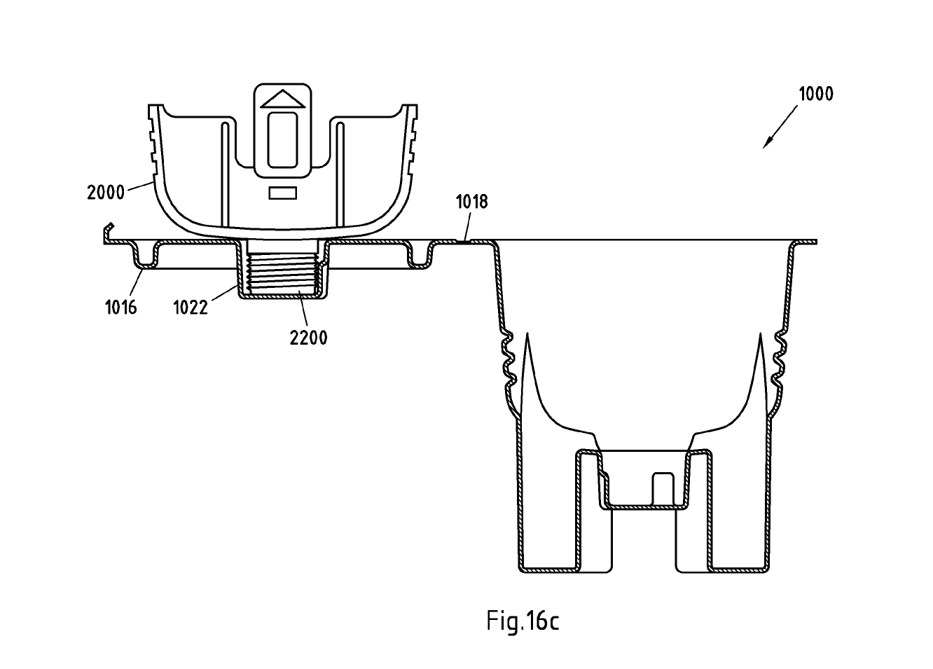

FIG. 16 a-d illustrate an exemplary embodiment of a blister package for a dispense interface according to the first, second and third aspect of the invention and a corresponding system;

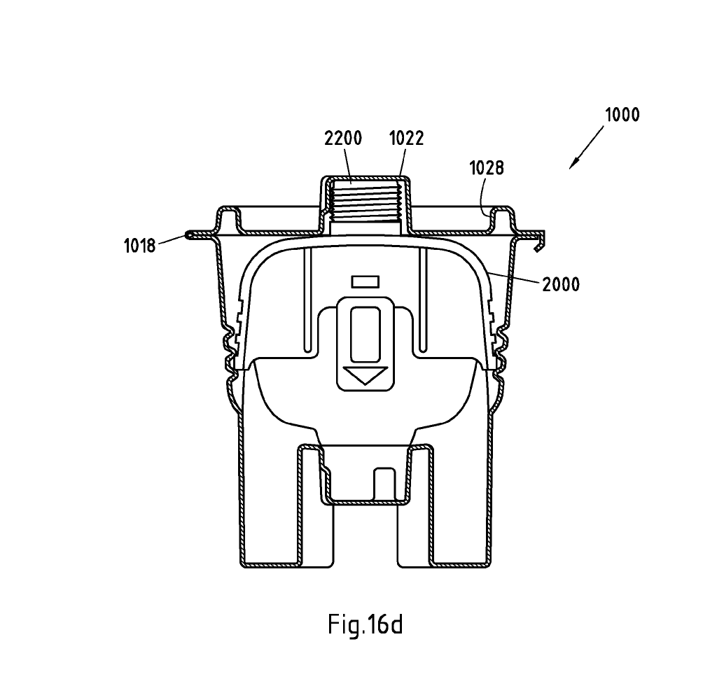

FIG. 17 illustrates a plurality of stacked blister packages from FIG. 16a-d;

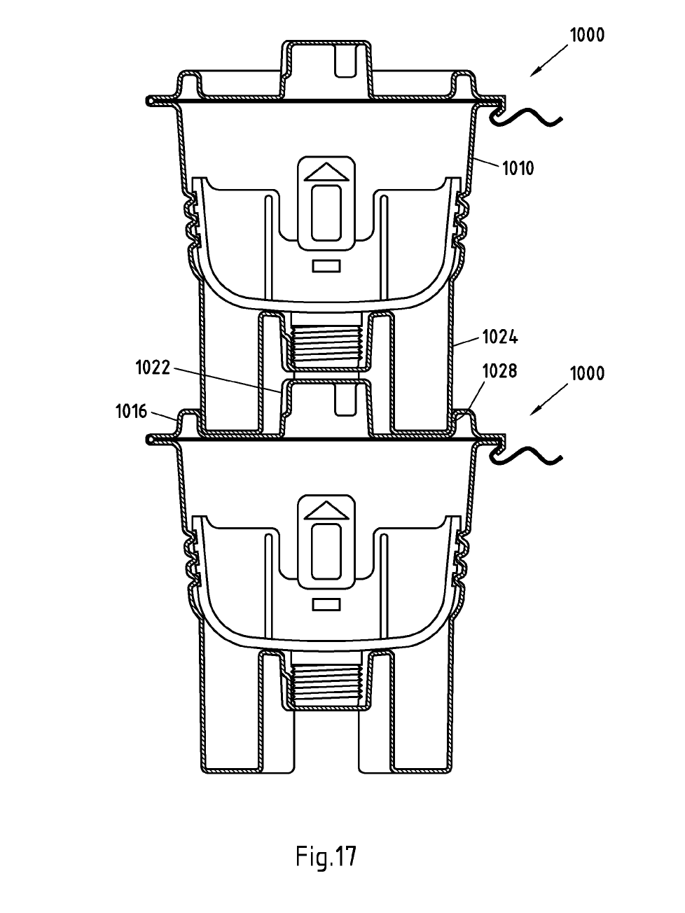

FIG. 18 a-c illustrate an exemplary embodiment of a blister package for a dispense interface according to the fourth aspect of the invention and a corresponding system;

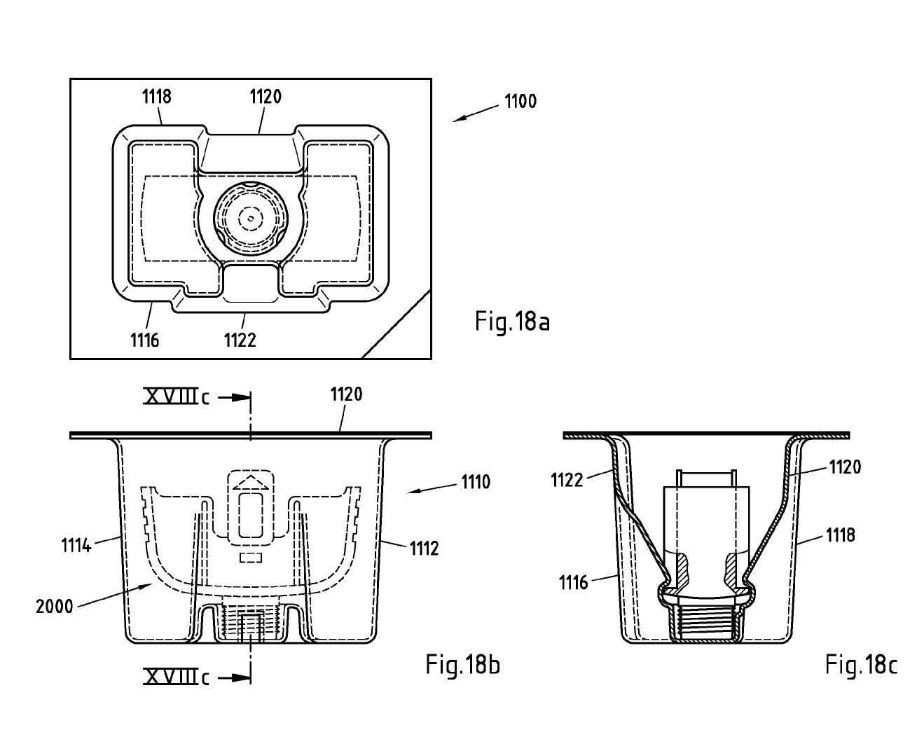

FIG. 19 illustrates a plurality of stacked blister packages from FIG. 18a-c;

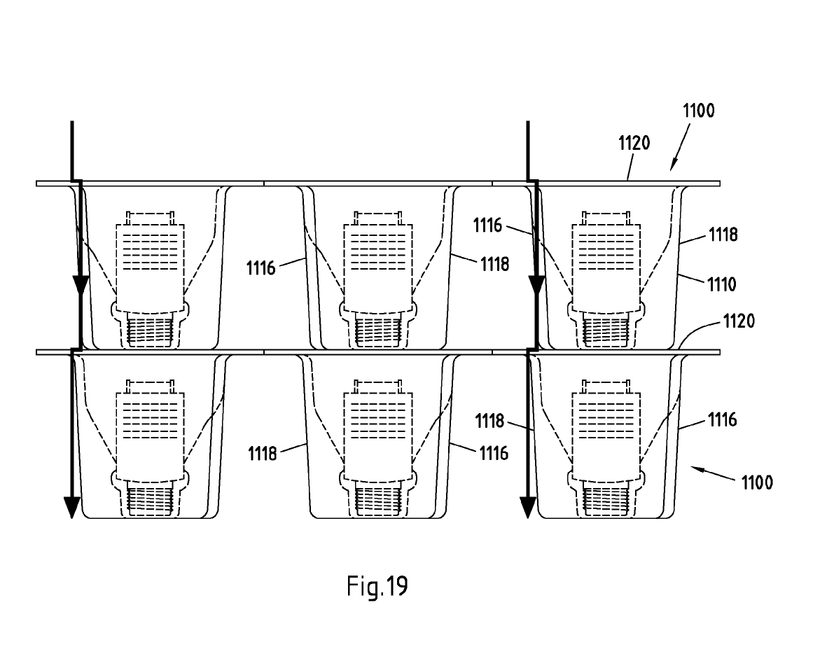

FIG. 20 a-b illustrate an exemplary embodiment of a blister package for a dispense interface according to the fifth and sixth aspect of the invention and a corresponding system;

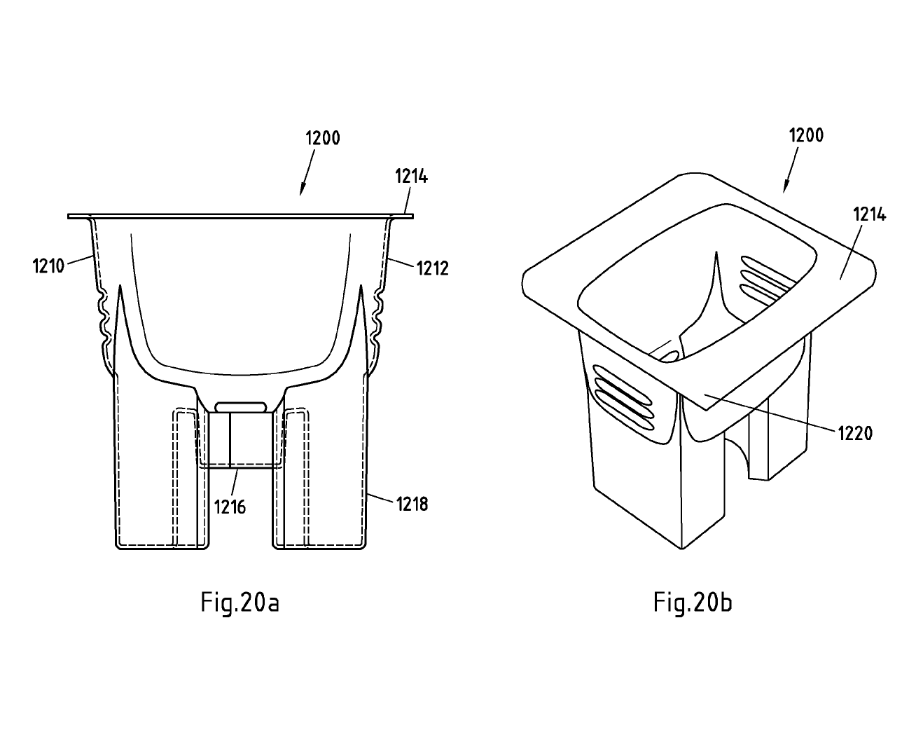

FIG. 21 shows an exemplary embodiment an outer package for a plurality of blister packages; and

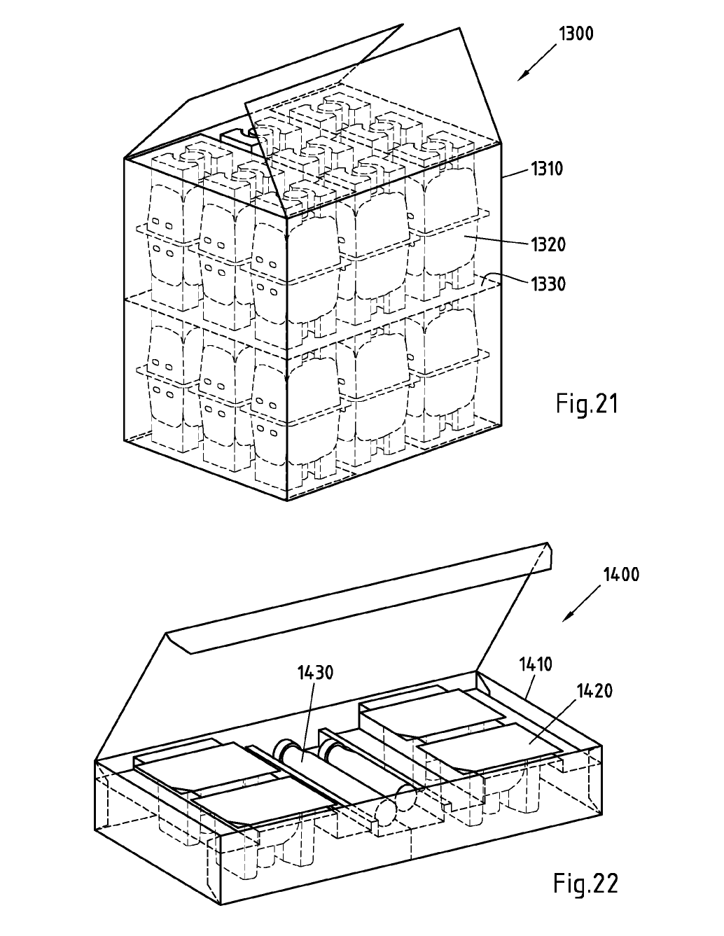

FIG. 22 shows another exemplary embodiment of an outer package for a plurality of blister packages.

The drug delivery device illustrated in FIG. 1 comprises a main body 14 that extends from a proximal end 16 to a distal end 15. At the distal end 15, a removable end cap or cover 18 is provided. This end cap 18 and the distal end 15 of the main body 14 work together to provide a snap fit or form fit connection so that once the cover 18 is slid onto the distal end 15 of the main body 14, this frictional fit between the cap and the main body outer surface 20 prevents the cover from inadvertently falling off the main body.

The main body 14 contains a micro-processor control unit, an electro-mechanical drive train, and at least two medicament reservoirs. When the end cap or cover 18 is removed from the device 10 (as illustrated in FIG. 1), a dispense interface 200 is mounted to the distal end 15 of the main body 14, and a dose dispenser (e.g., a needle assembly) is attached to the interface. The drug delivery device 10 can be used to administer a computed dose of a second medicament (secondary drug compound) and a variable dose of a first medicament (primary drug compound) through a single needle assembly, such as a double ended needle assembly.

The drive train may exert a pressure on the bung of each cartridge, respectively, in order to expel the doses of the first and second medicaments. For example, a piston rod may push the bung of a cartridge forward a pre-determined amount for a single dose of medicament. When the cartridge is empty, the piston rod is retracted completely inside the main body 14, so that the empty cartridge can be removed and a new cartridge can be inserted.

A control panel region 60 is provided near the proximal end of the main body 14. Preferably, this control panel region 60 comprises a digital display 80 along with a plurality of human interface elements that can be manipulated by a user to set and inject a combined dose. In this arrangement, the control panel region comprises a first dose setting button 62, a second dose setting button 64 and a third button 66 designated with the symbol “OK.” In addition, along the most proximal end of the main body, an injection button 74 is also provided (not visible in the perspective view of FIG. 1). The user interface of the drug delivery device may comprise additional buttons, such as a “menu” button, a “back” button, or a “light” button to switch on an illumination of the display.

The cartridge holder 40 can be removably attached to the main body 14 and may contain at least two cartridge retainers 50 and 52. Each retainer is configured so as to contain one medicament reservoir, such as a glass cartridge. Preferably, each cartridge contains a different medicament.

In addition, at the distal end of the cartridge holder 40, the drug delivery device illustrated in FIG. 1 includes a dispense interface 200. As will be described in relation to FIG. 4, in one arrangement, this dispense interface 200 includes a main outer body 212 that is removably attached to a distal end 42 of the cartridge housing 40. As can be seen in FIG. 1, a distal end 214 of the dispense interface 200 preferably comprises a needle hub 216. This needle hub 216 may be configured so as to allow a dose dispenser, such as a conventional pen type injection needle assembly, to be removably mounted to the drug delivery device 10.

Once the device is turned on, the digital display 80 shown in FIG. 1 illuminates and provides the user certain device information, preferably information relating to the medicaments contained within the cartridge holder 40. For example, the user is provided with certain information relating to both the primary medicament (Drug A) and the secondary medicament (Drug B).

As shown in FIG. 3, the first and second cartridge retainers 50, 52 may be hinged cartridge retainers. These hinged retainers allow user access to the cartridges. FIG. 3 illustrates a perspective view of the cartridge holder 40 illustrated in FIG. 1 with the first hinged cartridge retainer 50 in an open position. FIG. 3 illustrates how a user might access the first cartridge 90 by opening up the first retainer 50 and thereby having access to the first cartridge 90.

As mentioned above when discussing FIG. 1, a dispense interface 200 can be coupled to the distal end of the cartridge holder 40. FIG. 4 illustrates a flat view of the dispense interface 200 unconnected to the distal end of the cartridge holder 40. A dose dispenser or needle assembly 400 that may be used with the interface 200 is also illustrated and is provided in a protective outer cap 420.

In FIG. 5, the dispense interface 200 illustrated in FIG. 4 is shown coupled to the cartridge holder 40. The axial attachment means 48 between the dispense interface 200 and the cartridge holder 40 can be any known axial attachment means to those skilled in the art, including snap locks, snap fits, snap rings, keyed slots, and combinations of such connections. The connection or attachment between the dispense interface and the cartridge holder may also contain additional features (not shown), such as connectors, stops, splines, ribs, grooves, pips, clips and the like design features, that ensure that specific hubs are attachable only to matching drug delivery devices. Such additional features would prevent the insertion of a non-appropriate secondary cartridge to a non-matching injection device.

FIG. 5 also illustrates the needle assembly 400 and protective cover 420 coupled to the distal end of the dispense interface 200 that may be screwed onto the needle hub of the interface 200. FIG. 6 illustrates a cross sectional view of the double ended needle assembly 400 mounted on the dispense interface 200 in FIG. 5.

The needle assembly 400 illustrated in FIG. 6 comprises a double ended needle 406 and a hub 401. The double ended needle or cannula 406 is fixedly mounted in a needle hub 401. This needle hub 401 comprises a circular disk shaped element which has along its periphery a circumferential depending sleeve 403. Along an inner wall of this hub member 401, a thread 404 is provided. This thread 404 allows the needle hub 401 to be screwed onto the dispense interface 200 which, in one preferred arrangement, is provided with a corresponding outer thread along a distal hub. At a center portion of the hub element 401 there is provided a protrusion 402. This protrusion 402 projects from the hub in an opposite direction of the sleeve member. A double ended needle 406 is mounted centrally through the protrusion 402 and the needle hub 401. This double ended needle 406 is mounted such that a first or distal piercing end 405 of the double ended needle forms an injecting part for piercing an injection site (e.g., the skin of a user).

Similarly, a second or proximal piercing end 408 of the needle assembly 400 protrudes from an opposite side of the circular disc so that it is concentrically surrounded by the sleeve 403. In one needle assembly arrangement, the second or proximal piercing end 408 may be shorter than the sleeve 403 so that this sleeve to some extent protects the pointed end of the back sleeve. The needle cover cap 420 illustrated in FIGS. 4 and 5 provides a form fit around the outer surface 403 of the hub 401.

Referring now to FIG. 4 to 11, one preferred arrangement of this interface 200 will now be discussed. In this one preferred arrangement, this interface 200 comprises:

a. a main outer body 210,
b. an first inner body 220,
c. a second inner body 230,
d. a first piercing needle 240,
e. a second piercing needle 250,
f. a valve seal 260, and
g. a septum 270.

The main outer body 210 comprises a main body proximal end 212 and a main body distal end 214. At the proximal end 212 of the outer body 210, a connecting member is configured so as to allow the dispense interface 200 to be attached to the distal end of the cartridge holder 40. Preferably, the connecting member is configured so as to allow the dispense interface 200 to be removably connected the cartridge holder 40. In one preferred interface arrangement, the proximal end of the interface 200 is configured with an upwardly extending wall 218 having at least one recess. For example, as may be seen from FIG. 8, the upwardly extending wall 218 comprises at least a first recess 217 and a second recess 219.

Preferably, the first and the second recesses 217, 219 are positioned within this main outer body wall so as to cooperate with an outwardly protruding member located near the distal end of the cartridge housing 40 of the drug delivery device 10. For example, this outwardly protruding member 48 of the cartridge housing may be seen in FIGS. 4 and 5. A second similar protruding member is provided on the opposite side of the cartridge housing. As such, when the interface 200 is axially slid over the distal end of the cartridge housing 40, the outwardly protruding members will cooperate with the first and second recess 217, 219 to form an interference fit, form fit, or snap lock. Alternatively, and as those of skill in the art will recognize, any other similar connection mechanism that allows for the dispense interface and the cartridge housing 40 to be axially coupled could be used as well.

The main outer body 210 and the distal end of the cartridge holder 40 act to form an axially engaging snap lock or snap fit arrangement that could be axially slid onto the distal end of the cartridge housing. In one alternative arrangement, the dispense interface 200 may be provided with a coding feature so as to prevent inadvertent dispense interface cross use. That is, the inner body of the hub could be geometrically configured so as to prevent an inadvertent cross use of one or more dispense interfaces.

A mounting hub is provided at a distal end of the main outer body 210 of the dispense interface 200. Such a mounting hub can be configured to be releasably connected to a needle assembly. As just one example, this connecting means 216 may comprise an outer thread that engages an inner thread provided along an inner wall surface of a needle hub of a needle assembly, such as the needle assembly 400 illustrated in FIG. 6. Alternative releasable connectors may also be provided such as a snap lock, a snap lock released through threads, a bayonet lock, a form fit, or other similar connection arrangements.

The dispense interface 200 further comprises a first inner body 220. Certain details of this inner body are illustrated in FIG. 8-11. Preferably, this first inner body 220 is coupled to an inner surface 215 of the extending wall 218 of the main outer body 210. More preferably, this first inner body 220 is coupled by way of a rib and groove form fit arrangement to an inner surface of the outer body 210. For example, as can be seen from FIG. 9, the extending wall 218 of the main outer body 210 is provided with a first rib 213a and a second rib 213b. This first rib 213a is also illustrated in FIG. 10. These ribs 213a and 213b are positioned along the inner surface 215 of the wall 218 of the outer body 210 and create a form fit or snap lock engagement with cooperating grooves 224a and 224b of the first inner body 220. In a preferred arrangement, these cooperating grooves 224a and 224b are provided along an outer surface 222 of the first inner body 220.

In addition, as can be seen in FIG. 8-10, a proximal surface 226 near the proximal end of the first inner body 220 may be configured with at least a first proximally positioned piercing needle 240 comprising a proximal piercing end portion 244. Similarly, the first inner body 220 is configured with a second proximally positioned piercing needle 250 comprising a proximally piercing end portion 254. Both the first and second needles 240, 250 are rigidly mounted on the proximal surface 226 of the first inner body 220.

Preferably, this dispense interface 200 further comprises a valve arrangement. Such a valve arrangement could be constructed so as to prevent cross contamination of the first and second medicaments contained in the first and second reservoirs, respectively. A preferred valve arrangement may also be configured so as to prevent back flow and cross contamination of the first and second medicaments.

In one preferred system, dispense interface 200 includes a valve arrangement in the form of a valve seal 260. Such a valve seal 260 may be provided within a cavity 231 defined by the second inner body 230, so as to form a holding chamber 280. Preferably, cavity 231 resides along an upper surface of the second inner body 230. This valve seal comprises an upper surface that defines both a first fluid groove 264 and second fluid groove 266. For example, FIG. 9 illustrates the position of the valve seal 260, seated between the first inner body 220 and the second inner body 230. During an injection step, this seal valve 260 helps to prevent the primary medicament in the first pathway from migrating to the secondary medicament in the second pathway, while also preventing the secondary medicament in the second pathway from migrating to the primary medicament in the first pathway. Preferably, this seal valve 260 comprises a first non-return valve 262 and a second non-return valve 268. As such, the first non-return valve 262 prevents fluid transferring along the first fluid pathway 264, for example a groove in the seal valve 260, from returning back into this pathway 264. Similarly, the second non-return valve 268 prevents fluid transferring along the second fluid pathway 266 from returning back into this pathway 266.

Together, the first and second grooves 264, 266 converge towards the non-return valves 262 and 268 respectively, to then provide for an output fluid path or a holding chamber 280. This holding chamber 280 is defined by an inner chamber defined by a distal end of the second inner body both the first and the second non return valves 262, 268 along with a pierceable septum 270. As illustrated, this pierceable septum 270 is positioned between a distal end portion of the second inner body 230 and an inner surface defined by the needle hub of the main outer body 210.

The holding chamber 280 terminates at an outlet port of the interface 200. This outlet port 290 is preferably centrally located in the needle hub of the interface 200 and assists in maintaining the pierceable seal 270 in a stationary position. As such, when a double ended needle assembly is attached to the needle hub of the interface (such as the double ended needle illustrated in FIG. 6), the output fluid path allows both medicaments to be in fluid communication with the attached needle assembly.

The hub interface 200 further comprises a second inner body 230. As can be seen from FIG. 9, this second inner body 230 has an upper surface that defines a recess, and the valve seal 260 is positioned within this recess. Therefore, when the interface 200 is assembled as shown in FIG. 9, the second inner body 230 will be positioned between a distal end of the outer body 210 and the first inner body 220. Together, second inner body 230 and the main outer body hold the septum 270 in place. The distal end of the inner body 230 may also form a cavity or holding chamber that can be configured to be fluid communication with both the first groove 264 and the second groove 266 of the valve seal.

Axially sliding the main outer body 210 over the distal end of the drug delivery device attaches the dispense interface 200 to the multi-use device. In this manner, a fluid communication may be created between the first needle 240 and the second needle 250 with the primary medicament of the first cartridge and the secondary medicament of the second cartridge, respectively.

FIG. 11 illustrates the dispense interface 200 after it has been mounted onto the distal end 42 of the cartridge holder 40 of the drug delivery device 10 illustrated in FIG. 1. A double ended needle 400 is also mounted to the distal end of this interface. The cartridge holder 40 is illustrated as having a first cartridge containing a first medicament and a second cartridge containing a second medicament.

When the interface 200 is first mounted over the distal end of the cartridge holder 40, the proximal piercing end 244 of the first piercing needle 240 pierces the septum of the first cartridge 90 and thereby resides in fluid communication with the primary medicament 92 of the first cartridge 90. A distal end of the first piercing needle 240 will also be in fluid communication with a first fluid path groove 264 defined by the valve seal 260.

Similarly, the proximal piercing end 254 of the second piercing needle 250 pierces the septum of the second cartridge 100 and thereby resides in fluid communication with the secondary medicament 102 of the second cartridge 100. A distal end of this second piercing needle 250 will also be in fluid communication with a second fluid path groove 266 defined by the valve seal 260.

FIG. 11 illustrates a preferred arrangement of such a dispense interface 200 that is coupled to a distal end 15 of the main body 14 of drug delivery device 10. Preferably, such a dispense interface 200 is removably coupled to the cartridge holder 40 of the drug delivery device 10.

As illustrated in FIG. 11, the dispense interface 200 is coupled to the distal end of a cartridge housing 40. This cartridge holder 40 is illustrated as containing the first cartridge 90 containing the primary medicament 92 and the second cartridge 100 containing the secondary medicament 102. Once coupled to the cartridge housing 40, the dispense interface 200 essentially provides a mechanism for providing a fluid communication path from the first and second cartridges 90, 100 to the common holding chamber 280. This holding chamber 280 is illustrated as being in fluid communication with a dose dispenser. Here, as illustrated, this dose dispenser comprises the double ended needle assembly 400. As illustrated, the proximal end of the double ended needle assembly is in fluid communication with the chamber 280.

In one preferred arrangement, the dispense interface is configured so that it attaches to the main body in only one orientation, that is it is fitted only one way round. As such as illustrated in FIG. 11, once the dispense interface 200 is attached to the cartridge holder 40, the primary needle 240 can only be used for fluid communication with the primary medicament 92 of the first cartridge 90 and the interface 200 would be prevented from being reattached to the holder 40 so that the primary needle 240 could now be used for fluid communication with the secondary medicament 102 of the second cartridge 100. Such a one way around connecting mechanism may help to reduce potential cross contamination between the two medicaments 92 and 102.

FIG. 12a-c illustrate an exemplary embodiment of a blister package 500 for a dispense interface 2000 according to the first aspect of the invention and a corresponding system. FIG. 12a shows a view from below, FIG. 12b shows a longitudinal sectional view of blister package 500 corresponding to section plane XIIb indicated in FIG. 12a, and FIG. 12c shows a transversal sectional view of blister package 500 corresponding to section plane XIIc indicated in FIG. 12b. In FIGS. 12b and 12c, dispense interface 2000 is shown in plane view for simplicity.

The blister package 500 is configured for packaging dispense interface 2000 which may for example be identical or similar to dispense interface 200 depicted in FIG. 4. Together, blister package 500 and dispense interface 2000 form a system with blister package 500 being adapted to dispense interface 2000.

Dispense interface 2000 comprises a main body part 2100 with a main body proximal end 2110, a main body distal end 2120 and a mounting hub 2200 having an outer thread 2210 for mounting thereto a needle hub such as the needle hub of needle assembly 400 shown in FIG. 6. The main body part 2100 of dispense interface 2000 may comprise gripping grooves 2130 on two opposite sides to facilitate handling of the dispense interface 2000. The main body part 2100 further comprises a rim 2140 extending on both sides of the dispense interface 2000 as illustrated in FIG. 12c.

Blister package 500 comprises a blister 510 having a cavity portion 512 and a flange 514. Cavity portion 512 defines a cavity 516 which is configured to accommodate dispense interface 2000. The blister 510 may for example be made of a transparent plastic and may have been produced by injection moulding/vacuum forming. The blister package 500 further comprises a lid 520 which is bonded to flange 514 of the blister 510 by means of an adhesive such as glue and therewith seals the opening of blister 510 surrounded by flange 514. Preferably, lid 520 comprises a peel-off portion 530, in which lid 520 is not bonded to flange 514, so that a user may easily grab lid 520 in order to peel off the lid 520 from flange 514 to open the blister package 500.

The cavity portion 512 comprises two undercut portions 522 configured to interact with rim 2140 of dispense interface 2000 for locking dispense interface 2000 within cavity 516. The undercut portions 522 each comprise a bulge 524 having a first face 526 facing the lid 520 and a second face 528 facing away from lid 520, wherein the first face 526 is inclined with respect to the direction perpendicular to the plane of lid 520. When dispense interface 2000 is being inserted into cavity 516, main body distal end 2120 interacts with the second faces 528 so that bulges 524 are elastically pushed to the side giving way to completely insert dispense interface 2000 into cavity 516. After rim 2140 has passed bulges 524, bulges 524 snap back and thereby lock dispense interface 2000 within the cavity 516 by interaction of rim 2140 with second face 528 forming an undercut of the cavity portion 512 as illustrated in FIG. 12c. Dispense interface 2000 is then securely locked within blister package 500 so that sharp elements of dispense interface 2000 such as needles (not shown) are held back from lid 520 even if blister package 500 is rotated around to a bottom-up position.

FIG. 13 illustrates an example arrangement for stacking a plurality of blister packages 500 shown in FIG. 12a-c in a lateral view. The blister packages 500 are stacked up in rows, wherein the blister packages 500 in a first row have a first orientation and wherein the blister packages 500 in the second row being situated above the first row having a second orientation in which the blister packages are rotated around a vertical axis by about 90° with reference to the first row. Above the second row, there may be a third row (not shown) with blister packages 500 being oriented as in the first row and so on.

It was found that this stacking arrangement guides at least part of the compression forces (indicated by thick arrows) exerted from blister packages 500 from the second (or higher) row onto an according blister package 500 from the first row along vertical portions of the cavity portions 510, so that lid 520 is relieved from at least part of the compression force and therefore the risk of lid rupture is reduced.

FIG. 14a-c illustrate another exemplary embodiment of a blister package 700 for a dispense interface 2000 according to the first and second aspect of the invention and a corresponding system. FIG. 14a shows a longitudinal sectional view of blister package 700 corresponding to section plane XIVa indicated in FIG. 14a; FIG. 14b shows a side view seen from the left in FIG. 14a; and FIG. 14c shows a perspective view of a detail of the bottom of blister package 700. Dispense interface 2000 is shown in plane view in FIG. 14a for simplicity.

Like blister package 500, also blister package 700 comprises a blister 710 having a cavity portion 712 and a flange and comprises a lid 720 bonded to the flange. Blister 710 comprises a cavity portion 712 having a lateral portion 714 laterally enclosing a main body part 2100 of dispense interface 2000 being accommodated within blister package 700. The lateral portion 714 comprises two opposing faces 716, 718, wherein each of said faces 716, 718 comprises two snap fits 722, 724 configured to interact with the proximal end 2110 of main body part 2100. By means of these snap fits 722, 724 the dispense interface is securely locked within the cavity of cavity portion 712.

Cavity portion 712 further comprises a distal portion 726 (FIG. 14c) configured to accommodate a mounting hub 2200 of dispense interface 2000, wherein the distal portion 726 comprises radially inwardly directed clamping portions 728 configured to interact with outer thread 2210 of mounting hub 2200 of dispense interface 2000. By means of the clamping portions 728 the mounting hub 2200 is securely fixed within the bottom portion 726, so that on the one hand dispense interface 2000 is securely fixed within the cavity of cavity portion 712 and on the other hand central alignment of the dispense interface is facilitated when inserting dispense interface 2000 into cavity portion 712. Together, blister package 700 and dispense interface 2000 form a system with blister package 700 being adapted to dispense interface 2000.

FIG. 15 a-b illustrate an exemplary embodiment of a blister package 800 for a dispense interface 2000 according to the first and second aspect of the invention and a corresponding system. FIG. 15a shows a longitudinal sectional view of blister package 800 and FIG. 15b shows a side view corresponding to the side view of FIG. 14b from the left of FIG. 15a. Dispense interface 2000 is shown in plane view in FIG. 15a for simplicity.

Like blister packages 500 and 700, also blister package 800 comprises a blister 810 having a cavity portion 812 and a flange and comprises a lid 820 bonded to the flange. Cavity portion 812 of blister 810 comprises a lateral portion 814 configured to laterally enclose a main body part 2100 of dispense interface 2000, wherein two opposing faces 816, 818 of the lateral portion 814 each comprises a number of undercut portions 822 configured to interact with corresponding gripping grooves 2130 of the main body part 2100 of dispense interface 2000. By means of undercut portions 822 being adapted to the gripping grooves 2130 of dispense interface 2000, the dispense interface 2000 may be fixed securely within blister package 800 and in particular held back from lid 820, so that sharp elements of the dispense interface 2000 cannot damage lid 820 when turning blister package 800 bottom-up. Cavity portion 814 further comprises a bottom portion 824 configured like bottom portion 728 of blister package 700 shown in FIG. 16 to accommodate and fixate mounting hub 2200 of dispense interface 2000.

FIG. 16a-d illustrate an exemplary embodiment of a blister package 1000 for a dispense interface 2000 according to the first, the second and the third aspect of the invention and a corresponding system in longitudinal sectional views of blister package 1000 with dispense interface 2000 being shown in plane view for simplicity. Furthermore, FIG. 16a-d illustrate a possible sequence for using blister package 1000.

Like the other blister packages described above, also blister package 1000 comprises a blister 1010 and a lid 1020. The blister 1010 comprises a cavity portion 1012, a flange 1014 and a cap portion 1016 being pivotly connected to flange 1014 by an integral hinge 1018, so that cap portion 1016 is movable between a closed position (FIG. 16a) covering the cavity portion 1012 and an open position (FIG. 16b). The cap portion 1016 further comprises a reception portion 1022 configured to accommodated a mounting hub 2200 of dispense interface 2000. Lid 1020 is bonded to the flange 1014, thereby sealing the cavity within cavity portion 1012.

A dispense interface 2000 is preferably stored or provided to a user in a condition as shown in FIG. 16a, i.e. while being placed in blister package 1000 and therewith being hermetically sealed from the environment by cavity portion 1012 and lid 1020, so that sterility of dispense interface 2000 may be guaranteed over a sufficient amount of time.

If at a time a user would like to use dispense interface 2000 in order to mount it to a medicament ejection device, he may move the cap portion 1016 to the open position as shown in FIG. 16b, peel off lid 1020 and take dispense interface 2000 out of the blister package 1000, for example by picking up the dispense interface 2000 with the device. In this way, the user doesn't need to touch the dispense interface 2000 directly, so that needle stick injuries can be avoided. At the same time, the steps of taking the dispense interface 2000 out of the blister package 1000 and connecting the dispense interface 2000 to the device are united into a single step.

Blister package 1000 may also be used to securely accommodate dispense interface 2000 after use, so that the risk of injury due to sharp elements of the dispense interface 2000 such as needles is reduced and the dispense interface 2000 may for example even be disposed with regular household waste instead of disposal in a special sharps bin.

To this end, the user may place the mounting hub 2200 of a used dispense interface 2000 into reception portion 1022 of cap portion 1016 as shown in FIG. 16c. Reception portion 1022 may be designed similar to distal portion 726 of blister package 700 shown in FIG. 14c and having according radially inwardly directed clamping portions to fixate the mounting hub 2200 of dispense interface 2000.

Dispense interface 2000 may then be enclosed in blister package 1000 by simply moving the cap portion 1016 to the closed position as shown in FIG. 16d and without the risk of injury due to sharp elements of the dispense interface. Fluids still remaining within the dispense interface 2000 may then flow out of the dispense interface 2000 and into the cavity provided by cavity portion 1012.

When the blister package 1000 is closed as shown in FIG. 16d, the user may also push the reception portion 1022, thereby releasing mounting hub 2200, so that dispense interface 2000 may move further down into the cavity provided by cavity portion 1012.

FIG. 17 illustrates an example arrangement for stacking a plurality of blister packages 1000 shown in FIG. 16a-d in a longitudinal sectional view of blister packages 1000. In FIG. 17, dispense interfaces 2000 are shown in plane view for simplicity. To this end, each blister packages 1000 comprises a holding portion 1024 extending from the bottom portion 1026 of cavity portion 1012, so that the blister packages 1000 may be stacked on top of each other despite the protruding reception portions 1022. Moreover, cap portion 1016 may be provided with indentations 1028 corresponding to the shape of holding portion 1024, so that the holding portion 1024 of a blister package 1000 may be inserted into the according indentations 1028 of a blister package 1000 below for a better alignment and improved fixation.

FIG. 18a-c illustrate an exemplary embodiment of a blister package 1100 for a dispense interface 2000 according to the fourth aspect of the invention and a corresponding system. FIG. 18a shows an entire view from below, FIG. 18b shows a side view, and FIG. 18c shows a transversal sectional view of blister package 1100 corresponding to section plane XVIIIc indicated in FIG. 18b. Dispense interface 2000 is shown in plane view in FIG. 18c for simplicity.

Blister package 1100 comprises a blister 1110 with a cavity portion 1112 and a flange, and a lid 1120 bonded to the flange. The cavity portion 1112 comprises a lateral portion 1114 configured to laterally disclose a dispense interface 2000 to be packaged in the blister package 1100. Blister package 1100 differs from blister package 500 shown in FIG. 12 in that the lateral portion 1114 comprises at least two opposing faces 1116, 1118, the outer shape of which is mirror asymmetrical. In the case of blister package 1100 this is realised in that the cavity portion 1112 in FIG. 18a has a substantially rectangular cross-section with an additional indentation 1120 on one face 1118 and an additional protrusion 1122 on the other face 1116. Indentation 1120 and protrusion 1122 break the mirror symmetry of lateral portion 1114 with respect to a vertical, in particular longitudinal, mirror plane through cavity portion 1112. This asymmetry may be exploited for stacking the blister packages 1100 in a way that reduces compressional forces acting on lid 1120.

FIG. 19 illustrates an example arrangement for stacking a plurality of blister packages 1100 shown in FIG. 18a-c in a side view. The blister packages 1100 are stacked up in rows, wherein the blister packages 1100 in a first row have a first orientation and the blister packages 1100 in the second row being situated above the first row have a second orientation in which the blister packages are rotated around a vertical axis by about 180° with reference to the first row. Above the second row, there may be a third row (not shown) with blister packages 1100 being oriented as in the first row and so on.

It was found that due to the mirror asymmetrical faces 1118, 1116 of the blister packages 1100 and with this stacking arrangement, compression forces (indicated by thick arrows) exerted from a blister package 1100 from the second (or higher) row onto an according blister package 1100 from the first row may be guided along vertical portions of the cavity portions 510, so that lid 520 is relieved from at least part of the compression forces and therefore the risk of lid rupture is reduced.

FIG. 20a-b illustrate an exemplary embodiment of a blister package for a dispense interface according to the fifth and sixth aspect of the invention. FIG. 20a shows a lateral view and FIG. 20b shows a perspective view. Blister package 1200 comprises a blister 1210 with a cavity portion 1212 and a flange 1214, and a lid (not shown) bonded to flange 1214. At least 50% of the flange 1214 has a width of at least 20 mm, so that it is easier for handicapped persons to handle the blister package 1200, in particular to peel off the lid (not shown) in order to open the blister package 1200. To this end, the width of flange 1214 is at least 20 mm in the peel-off region 1220, in which the lid is unbonded to flange 1214 and may be grabbed by a user to peel off the lid.

The bottom of the cavity of cavity portion 1212 defines a bottom portion 1216 of blister 1210 which is located opposite to the lid. Blister 1210 comprises a holding portion 1218 extending away from the lid and beyond bottom portion 1216. By means of this holding portion 1218 the total height of the blister package 1200 is increased by preferably 10%, more preferably at least 25% of the original height of the cavity of the cavity portion 1212. By increasing the blister height, handling of blister package 1200 may be made easier, in particular for handicapped persons. Blister package 1200 and an according dispense interface (not shown) together form a system with blister package 1200 preferably being adapted to the dispense interface.

FIG. 21 shows an exemplary embodiment of an outer package 1300 for a plurality of blister packages in half-transparent perspective view. The outer package 1300 comprises a box 1310 in which blister packages 1320 are aligned face to face, or rather lid to lid, i.e. with odd layers of blister packages being arranged bottom-down and with even layers of blister packages being arranged bottom-up. A plate 1330 for example of cardboard may be placed above two planes of blister packages 1320 in order to stabilize the stack before inserting another two layers of blister packages 1320 into box 1310. The blister packages 1320 may for example be similar to blister packages 500, 700 or 800.

FIG. 22 shows another exemplary embodiment of an outer package for a plurality of blister packages in half-transparent perspective view. The outer package 1400 comprises a box 1410 in which a number of blister packages 1420 are arranged. The blister packages 1420 may be fixated within the box 1410 by means of according fixation means. The box 1410 further comprises a reception for receiving cartridges 1430 to be used in a medicament ejection device together with the dispense interface. In such a way the outer package 1400 may for example be provided as a unit including both, drug cartridges 1430 and blister packages 1420 with dispense interfaces to be used with a particular medicament ejection device.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta [symbol] decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

1. A blister package (500, 700, 800, 1000, 1100, 1200, 1320, 1420) for packaging a dispense interface (200, 2000) of a medicament ejection device, comprising
a blister (510, 710, 810, 1010, 1110, 1210) having a cavity portion (512, 712, 812, 1012, 1112, 1212) and a flange (514, 1014, 1214), the cavity portion (512, 712, 812, 1012, 1112, 1212) defining a cavity (516) and being configured to hold a dispense interface (200, 2000) of a medicament ejection device within the cavity (516), and
a lid (520, 720, 1020, 1120, 820) bonded to the flange (514, 1014, 1214) of said blister (510, 710, 810, 1010, 1110, 1210),
characterized in that
the cavity portion (512, 712, 812, 1012, 1112, 1212) of said blister (510, 710, 810, 1010, 1110, 1210) comprises at least one undercut portion configured to interact with a corresponding part of the dispense interface (200, 2000) to be packaged in the blister package (500, 700, 800, 1000, 1100, 1200, 1320, 1420) to lock the dispense interface (200, 2000) within the cavity (516).

2. The blister package according to claim 1,
wherein the cavity portion (512, 712, 812, 1012, 1112, 1212) comprises a lateral portion (714, 814, 1114) configured to laterally enclose a main body part (2100) of a dispense interface (200, 2000) to be packaged in the blister package (500, 700, 800, 1000, 1100, 1200, 1320, 1420) and wherein two opposing faces (716, 718, 816, 818, 1116, 1118) of the lateral portion (714, 814, 1114) each comprise at least one snap-fit (722, 724) configured to interact with a proximal end (2110) of the dispense interface (200, 2000).

3. The blister package according claim 1 or 2,
wherein the cavity portion (512, 712, 812, 1012, 1112, 1212) comprises a lateral portion (714, 814, 1114) configured to laterally enclose a main body part (2100) of a dispense interface (200, 2000) to be packaged in the blister package (500, 700, 800, 1000, 1100, 1200, 1320, 1420) and wherein two opposing faces (716, 718, 816, 818, 1116, 1118) of the lateral portion (714, 814, 1114) each comprise at least one undercut portion configured to interact with corresponding gripping grooves (2130) on the main body part (2100) of the dispense interface (200, 2000).

4. The blister package according to any one of claims 1 to 3,
wherein the undercut portion of the cavity portion (512, 712, 812, 1012, 1112, 1212) comprises a bulge (524) having a first face (526) facing the lid (520, 720, 1020, 1120, 820) and a second face (528) facing away from the lid (520, 720, 1020, 1120, 820), said first face (526) forming the undercut surface and said second face (528) being inclined to guide a part of the dispense interface (200, 2000) to be packaged in the blister package (500, 700, 800, 1000, 1100, 1200, 1320, 1420) over the bulge (524).

5. A blister package (500, 700, 800, 1000, 1100, 1200, 1320, 1420) for packaging a dispense interface (200, 2000) of a medicament ejection device,
with a blister (510, 710, 810, 1010, 1110, 1210) having a cavity portion (512, 712, 812, 1012, 1112, 1212) and a flange (514, 1014, 1214), the cavity portion (512, 712, 812, 1012, 1112, 1212) defining a cavity (516) and being configured to hold a dispense interface (200, 2000) of a medicament ejection device within the cavity (516), and
with a lid (520, 720, 1020, 1120, 820) bonded to the flange (514, 1014, 1214) of said blister (510, 710, 810, 1010, 1110, 1210),
characterized in that
the cavity portion (512, 712, 812, 1012, 1112, 1212) comprises a distal portion (726) configured to accommodate a mounting hub (2200) of the dispense interface (200, 2000) to be packaged in the blister package (500, 700, 800, 1000, 1100, 1200, 1320, 1420) and
the distal portion (726) comprises radial inwardly directed clamping portions (728) configured to interact with the mounting hub (2200) of the dispense interface (200, 2000).

6. A blister package (500, 700, 800, 1000, 1100, 1200, 1320, 1420) for packaging a dispense interface (200, 2000) of a medicament ejection device,
with a blister (510, 710, 810, 1010, 1110, 1210) having a cavity portion (512, 712, 812, 1012, 1112, 1212) and a flange (514, 1014, 1214), the cavity portion (512, 712, 812, 1012, 1112, 1212) defining a cavity (516) and being configured to hold a dispense interface (200, 2000) of a medicament ejection device within the cavity (516), and
with a lid (520, 720, 1020, 1120, 820) bonded to the flange (514, 1014, 1214) of said blister (510, 710, 810, 1010, 1110, 1210),
characterized in that
the blister (510, 710, 810, 1010, 1110, 1210) comprises a cap portion (1016) being pivotably connected to the flange (514, 1014, 1214) by an integral hinge so that the cap portion (1016) is movable between a closed position covering the cavity portion (512, 712, 812, 1012, 1112, 1212) and an open position, and
the cap portion (1016) comprises a reception portion (1022) configured to accommodate a mounting hub (2200) of the dispense interface (200, 2000) to be packaged in the blister package (500, 700, 800, 1000, 1100, 1200, 1320, 1420).

7. A blister package (500, 700, 800, 1000, 1100, 1200, 1320, 1420) for packaging a dispense interface (200, 2000) of a medicament ejection device,
with a blister (510, 710, 810, 1010, 1110, 1210) having a cavity portion (512, 712, 812, 1012, 1112, 1212) and a flange (514, 1014, 1214), the cavity portion (512, 712, 812, 1012, 1112, 1212) defining a cavity (516) and being configured to hold a dispense interface (200, 2000) of a medicament ejection device within the cavity (516), and
with a lid (520, 720, 1020, 1120, 820) bonded to the flange (514, 1014, 1214) of said blister (510, 710, 810, 1010, 1110, 1210),
characterized in that
the cavity portion (512, 712, 812, 1012, 1112, 1212) comprises a lateral portion (714, 814, 1114) configured to laterally enclose a dispense interface (200, 2000) to be packaged in the blister package (500, 700, 800, 1000, 1100, 1200, 1320, 1420), and
the lateral portion (714, 814, 1114) comprises at least two opposing faces (716, 718, 816, 818, 1116, 1118), the outer shape of which is mirror asymmetrical.

8. A blister package (500, 700, 800, 1000, 1100, 1200, 1320, 1420) for packaging a dispense interface (200, 2000) of a medicament ejection device,
with a blister (510, 710, 810, 1010, 1110, 1210) having a cavity portion (512, 712, 812, 1012, 1112, 1212) and a flange (514, 1014, 1214), the cavity portion (512, 712, 812, 1012, 1112, 1212) providing a cavity (516) and being configured to hold a dispense interface (200, 2000) of a medicament ejection device within the cavity (516), and
with a lid (520, 720, 1020, 1120, 820) bonded to the flange (514, 1014, 1214) of said blister (510, 710, 810, 1010, 1110, 1210),
characterized in that
the blister (510, 710, 810, 1010, 1110, 1210) comprises a bottom portion (1216) being located opposite to the lid (520, 720, 1020, 1120, 820) and defining the bottom of the cavity (516) and
the blister (510, 710, 810, 1010, 1110, 1210) further comprises a holding portion (1024, 1218) extending away from the lid (520, 720, 1020, 1120, 820) and beyond bottom portion (1216).

9. A blister package (500, 700, 800, 1000, 1100, 1200, 1320, 1420) for packaging a dispense interface (200, 2000) of a medicament ejection device, comprising
a blister (510, 710, 810, 1010, 1110, 1210) having a cavity portion (512, 712, 812, 1012, 1112, 1212) and a flange (514, 1014, 1214), the cavity portion (512, 712, 812, 1012, 1112, 1212) providing a cavity (516) and being configured to hold a dispense interface (200, 2000) of a medicament ejection device within the cavity (516), and
a lid (520, 720, 1020, 1120, 820) bonded to the flange of said blister (510, 710, 810, 1010, 1110, 1210),
characterized in that
the lid (520, 720, 1020, 1120, 820) comprises a peel-off portion (530), in which the lid (520, 720, 1020, 1120, 820) is less strongly bonded or unbonded to the flange (514, 1014, 1214) and
the flange (514, 1014, 1214) has a width of at least 10 mm, preferably at least 15 mm, in particular at least 20 mm, at least in the region of the peel-off portion (530).

10. A blister package (500, 700, 800, 1000, 1100, 1200, 1320, 1420) for packaging a dispense interface (200, 2000) of a medicament ejection device,
wherein the blister package (500, 700, 800, 1000, 1100, 1200, 1320, 1420) is according to any one of claims 1 to 9 or any combination thereof.

11. A system comprising:
a dispense interface (200, 2000) of a medicament ejection device and
a blister package (500, 700, 800, 1000, 1100, 1200, 1320, 1420) according to any one of claims 1 to 10,
wherein the blister package (500, 700, 800, 1000, 1100, 1200, 1320, 1420) is adapted to the dispense interface (200, 2000).

12. The system according to claim 11,
wherein the dispense interface (200, 2000) is accommodated in the cavity (516) provided by the cavity portion (512, 712, 812, 1012, 1112, 1212) of the blister package (500, 700, 800, 1000, 1100, 1200, 1320, 1420).

13. The system according to claim 11 or 12,
wherein the dispense interface (200, 2000) comprises a main body part (2100) with a main body proximal end (2110) and a main body distal end (2120) and wherein a mounting hub (2200) is provided at the distal end (2120) of the main body part (2100), which mounting hub (2200) preferably comprises an outer thread (2210) configured to interact with an inner thread of a needle hub (401) of a needle assembly (400).
